(19) United States
(12) Patent Application Publication
(10) Pub. No.: US 2022/0040477 A1
(43) Pub. Date: Feb. 10, 2022

(54) MULTICHANNEL IONTOPHORETIC DEVICES FOR DENTAL AND DERMAL APPLICATIONS

(71) Applicant: HG MEDICAL TECHNOLOGIES LLC, Fort Collins, CO (US)

(72) Inventor: Julian Henley, Fort Collins, CO (US)

(21) Appl. No.: 17/413,596

(22) PCT Filed: Dec. 12, 2019
(86) PCT No.: PCT/US19/65898

§ 371 Date: Jun. 14, 2021

Related U.S. Application Data
(60) Provisional application No. 62/778,410, filed on Dec. 12, 2018.

(51) Int. Cl. A61N 1/30; A61N 1/04

(52) U.S. Cl. CPC A61N 1/303; A61N 1/0428

(57) ABSTRACT

An iontophoretic device including a multichannel driver connected to multiple electrodes, a medication reservoir positioned distal to the multiple electrodes and including multiple reservoir chambers, and a resistive barrier material at least partially surrounding one or more of the multiple electrodes and one or more of the multiple reservoir chambers, the resistive barrier material configured to provide a least resistive current flow path running from an active electrode to a tissue contact point most proximal to the active electrode.

### CROSS-REFERENCE TO RELATED APPLICATIONS

This application claims priority to U.S. provisional application No. 62/778,410 filed on Dec. 12, 2018, incorporated herein by reference in its entirety.

### BACKGROUND OF THE INVENTION

Iontophoresis is a well-established technique of enhancing the penetration of variety of soluble molecules into skin, tissue, and mucous membranes (see e.g. International Patent Application No. PCT/US16/67450 to Henley). It is based on the principle of applying electromotive force (i.e. voltage) to a conductive electrode in proximity to target tissue with an interposed reservoir containing a solubilized molecule. The desired molecule becomes a primary charge carrier as it moves from the interposed reservoir into the target tissue with which it is in contact. A counter electrode is applied at a distant location so that the tissue (e.g. skin or mucous membranes) are subjected to the voltage push and the desired molecule is therefore transported into the target tissue at an accelerated transfer as compared to topical application. In one aspect, iontophoretic applications can be described as a topical penetration amplifier, and can be used in combination with penetration enhancers such as DMSO, ultrasound, phonophoresis, electrophoresis, and micro needles or micro abrasion. Penetration enhancers mainly degrade the keratin barrier and make skin more penetrable.

Although iontophoresis has been shown to be a safe and effective process, it has limitations related to the amount of current flux that tissue can tolerate before areas of irritation, blistering, or burn occur at the electrode application site. The prior art describes safe limits of current flux for skin and tissue in the range of 1.2-1.6 ma/cm2. At the same time, it is desirable to have iontophoretic devices capable of driving more medication into skin or tissue. To accomplish this, larger electrodes have been constructed to cover a larger contact area and thereby allow for larger current to carry medication into tissue. However, this solution turned out to be problematic because tissue in contact with a larger electrode does not have uniform resistance and current preferentially flows through path of least resistance. This results in a preferential current flow through a smaller area of tissue (e.g. a lesion or skin rupture) rather than an even distribution. When this happens, a blister or burn is likely to occur since the current density within the smaller area exceeds the normative 1.6 ma/cm2.

One solution which at least partially addresses the issue of current distribution along a larger therapeutic area and safely delivering larger total carrier current over larger area is in the form of a multichannel iontophoretic system, described for example in U.S. Pat. No. 5,160,316 to Henley. Such systems describe a larger electrode composed of separately controlled current channels which assure controlled current dispersion over a wider tissue contact area. Each channel is driven by separate electronic circuits to assure wide dispersion and enhanced penetration of medicament. These wide field electrodes not only can cover a wide area of body without succumbing to “tunneling effects”, but provide sufficient skin penetration to function as a systemic drug delivery system. An isolated current loop generator is employed to feed current to the individual channels in the multichannel electrode via the plurality of individual current loops. Each current loop drives one band or channel in the multichannel electrode. It is disclosed that a 0.6 milliamp current flowing to each channel used within a wide field dispersion grounding electrode provides a safe level for operating the iontophoretic device. This level of current can help to minimize the tunneling effect of current flowing along the path of least resistance and concentrating in, for example, a lesion or skin rupture, resulting in a burn to the patient. This permits current to be distributed over the large area of the multichannel electrode to drive medicament through a patient's skin over a large dermal area. These systems partially address the blistering effect of single channel technology due to current following a path of least resistance and creating a contact burn related to current density exceeding tissue tolerance at contact spots of least resistance. However, greater safety margins are nonetheless desired to avoid any remaining tunneling effects or ununiform distribution of current, while further increasing the efficacy of medicament delivery.

What is needed in the art is an improved device that can provide additional safety margins and amplify topical penetration, all while avoiding the frequent pitfalls described above that afflict other conventional iontophoretic devices.

### SUMMARY OF THE INVENTION

In one embodiment, an iontophoretic device includes a multichannel driver connected to a plurality of electrodes, a medication reservoir positioned distal to the plurality of electrodes and comprising a plurality of reservoir chambers, and a resistive barrier material at least partially surrounding one or more of the plurality of electrodes and one or more of the plurality of reservoir chambers, the resistive barrier material configured to provide a least resistive current flow path running from an active electrode to a tissue contact point most proximal to the active electrode. In one embodiment, the resistive barrier material is a first material at least partially surrounding the one or more of the plurality of electrodes and second material at least partially surrounding the one or more of the plurality of reservoir chambers. In one embodiment, the plurality of electrodes are separated by the resistive barrier material. In one embodiment, the resistive barrier material is configured to allow current flow in a distal direction. In one embodiment, the resistive barrier material is configured to block current flow in a direction perpendicular to the distal direction. In one embodiment, each of the plurality of reservoir chambers are positioned directly below a different electrode. In one embodiment, each of the plurality of electrodes are configured to direct a current flow path through a single reservoir chamber before reaching a target treatment area. In one embodiment, the multichannel driver is configured to drive 0.6 milliamps or less of current through each channel. In one embodiment, the resistive barrier material is disposed within the medication reservoir such that during application, a flow path from an active electrode to a tissue contact point most proximal to an adjacent electrode is resistive. In one embodiment, the resistive barrier material is disposed within the medication reservoir such that during application, an inter-electrode flow path and an inter-channel flow path are both resistive. In one embodiment, the medication reservoir is constructed from a resistive barrier mesh comprising open cells configured to hold medication in form of a liquid, gel, or ointment. In one embodiment, the medication reservoir comprises a plurality of layers of open cell mesh. In one embodiment, the medication reservoir comprises a polymer open cell structure. In one embodiment, the medication reservoir comprises fibers with wicking properties. In one embodiment, inter-channel spaces are rendered resistive barrier by at least one of thermal cell collapse, nonconductor geometric infiltration or nonconductor structural isolation.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram of a current polarizing multichannel iontophoretic device according to one embodiment.

FIG. 2 is a diagram of a current polarizing multichannel iontophoretic device having an open cell chamber arrangement according to one embodiment.

FIGS. 3A and is a cross-sectional views of reservoir chamber geometry according to one embodiment, and FIG. 3B is a cross-sectional views of reservoir chamber geometry according to another embodiment.

FIG. 4 is a diagram of a current polarizing multichannel iontophoretic device having cell foam chambers according to one embodiment.

FIG. 5A is an image of a prototype of a dental applicator current polarizing multichannel iontophoretic device according to one embodiment, and FIG. 5B is an image of a prototype of a dental applicator current polarizing multichannel iontophoretic device according to another embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in multichannel iontophoretic devices for dental and dermal applications. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a multichannel iontophoretic devices for dental and dermal applications.

Embodiments of the device amplify topical penetration while avoiding the tunneling effect of current flowing along the path of least resistance, an issue which afflicts conventional iontophoretic devices. To improve both the safety and efficacy of multichannel iontophoretic devices, the multichannel application electrode described herein is designed to control the current at each location of the treatment region even if the tissue resistance path is variable. In one embodiment, a current polarization element is incorporated into the medication reservoir portion of the multichannel iontophoretic contact electrode. Current polarization in one aspect is achieved by making the path resistance lowest at the most proximal tissue contact area. This way, current flows directly from the electrode through the reservoir layer and into the most proximal tissue contact area. A higher resistance is also avoided by mitigating inter-channel current flow. In most cases, the medication reservoir is flexible to contour with contact tissue, yet it can be wide for larger area application. Embodiments of the device incorporate current polarization to ensure that the preferred current path is minimized in lateral or inter-electrode flow by virtue of encountering a higher resistance in the non-preferred lateral direction. Embodiments of the device typically never exceed 0.6 ma/cm2 for providing an improved safety margin. In one embodiment, the interposed reservoir is an absorbent fiber material substrate with wax ink printed on it in a specific pattern to encompass each electrode. The wax barrier creates a resistive barrier to inter-channel conductivity.

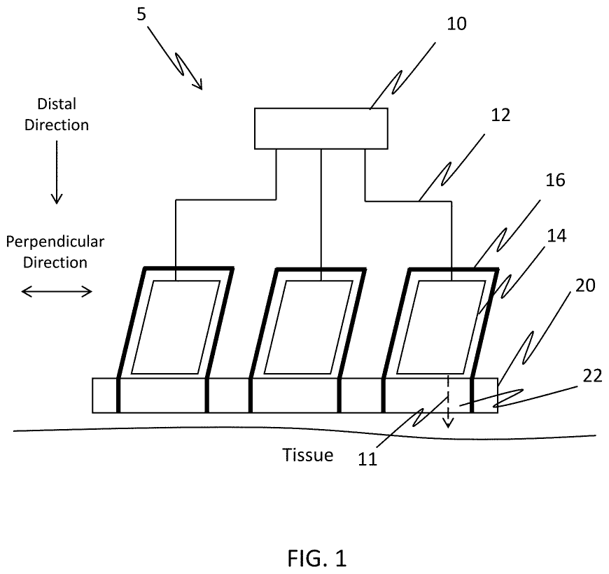

With reference now to FIG. 1, according to one embodiment, an iontophoretic device 5 includes a driver 10 and drive lines 12 connected to separate electrodes 14 that are conductively isolated by an inert resistive barrier material 16, such as a high resistance/partial conductor or nonconductive material. The medication reservoir 20 forms chambers 22 to retain the medication in its aqueous or gel form. The chambers 22 are interposed between the active conductive electrode 14 and the target tissue. The chambers 22 are also isolated by the resistive barrier material 16 and configured to provide a direct conductive pathway 11 from a corresponding electrode 14 to the tissue treatment area. The conductive path between the electrode 14 and tissue via the corresponding chamber 22 provides a current polarizing effect by providing a least resistive current flow path 11 running from the active electrode directly to the tissue contact point most proximal to the active electrode. Each channel's electrode and corresponding chamber(s) are conductively isolated from electrode and corresponding chamber(s) of other channels. Side-to-side conductivity is reduced by the higher-resistance resistive barrier provided by the resistive barrier material 16. The path of least resistance is the path directly to the proximal-most tissue, which promotes a greater direct conduction of charge via charged medication molecules. Additional embodiments of iontophoretic devices having a lower resistance in the distal direction and a higher resistance in the perpendicular direction are described herein. Resistive barrier materials can be of types known in the art, such as resistive barrier polymers, plastics, waxes, rubber, and other resistive barrier materials described herein.

Accordingly, in one embodiment, an iontophoretic device includes a multichannel driver connected to multiple electrodes, and a medication reservoir positioned distal to the electrodes. The medication reservoir includes multiple reservoir chambers. A resistive barrier material at least partially surrounds one or more of the electrodes and one or more of the reservoir chambers. The resistive barrier material is configured to provide a least resistive current flow path running from an active electrode to a tissue contact point most proximal to the active electrode. In one embodiment, the resistive barrier material is a first material at least partially surrounding the one or more of the plurality of electrodes and second material at least partially surrounding the one or more of the plurality of reservoir chambers. In one embodiment, the electrodes are separated by the resistive barrier material. In one embodiment, the resistive barrier material allows current flow in a distal direction. In one embodiment, the resistive barrier material blocks current flow in a direction perpendicular to the distal direction. In one embodiment, each of the reservoir chambers are positioned directly below a different electrode. In one embodiment, each of the electrodes direct a current flow path through a single reservoir chamber before reaching a target treatment area. In one embodiment, the multichannel driver drives 0.6 milliamps or less of current through each channel. In one embodiment, during application, a flow path from an active electrode to a tissue contact point most proximal to an adjacent electrode is resistive. In one embodiment, during application, an inter-electrode flow path and an inter-channel flow path are both resistive. In one embodiment, the medication reservoir is constructed from a resistive barrier mesh including open cells to hold medication in form of a liquid, gel, or ointment. In one embodiment, the medication reservoir includes a plurality of layers of open cell mesh. In one embodiment, the medication reservoir includes a polymer open cell structure. In one embodiment, the medication reservoir includes fibers with wicking properties. In one embodiment, inter-channel spaces are rendered resistive barrier by at least one of thermal cell collapse, nonconductor geometric infiltration or nonconductor structural isolation.

In one embodiment, the electrode forms a closed circuit through the patient's body when current passes therethrough which promotes the penetration or absorption of an ionic medicament contained in the adjacent medication reservoir. The polarity of the working electrode can be selected based upon the polarity of the medicament to be administered. In one embodiment, the electrode includes a flexible sheet or film forming a conductive matrix having a current distributing conductive layer, such as a metallic foil, a conductive rubber or resin film, carbon film or other conductive coating or electro-dispersive material (see e.g. U.S. Pat. No. 5,658,247 to Henley). The conductive matrix can be flexible so that it may be contoured to the body area on which it is placed and still cover a relatively wide area. A grounding electrode (not shown) employed with the multichannel electrode can be implemented to cover an area of skin which is similar in size to the area covered by primary electrode.

A ribbon connector can be used to connect an electrical power source to the multichannel electrode for delivering the electrical current by means of the drive lines that form the individual electrically conductive channels in the conductive matrix. Each channel in the iontophoretic array preferably carries no more than 0.6 milliamps. The amount of current that flows to each channel is controlled by a control circuit, which along with the polarization effects described herein help to eliminate a tunneling effect. This prevents the flow of current along the path of least resistance through a lesion or skin rupture, for example, resulting in a burn to the patient at that location. The multichannel electrode can employ a circuit pattern etched such as by laser or photoetching onto, for example, a metal coated plastic sheet with each channel isolated to facilitate dispersion over a broad surface area. Each channel formed by the drive wires can be electrically driven simultaneously or in a sequential multiplex fashion. The use of simultaneous or parallel electrical current to each drive wire in the array could be employed, for example, in the application of medicament to burns where a wide area of dispersion is required.

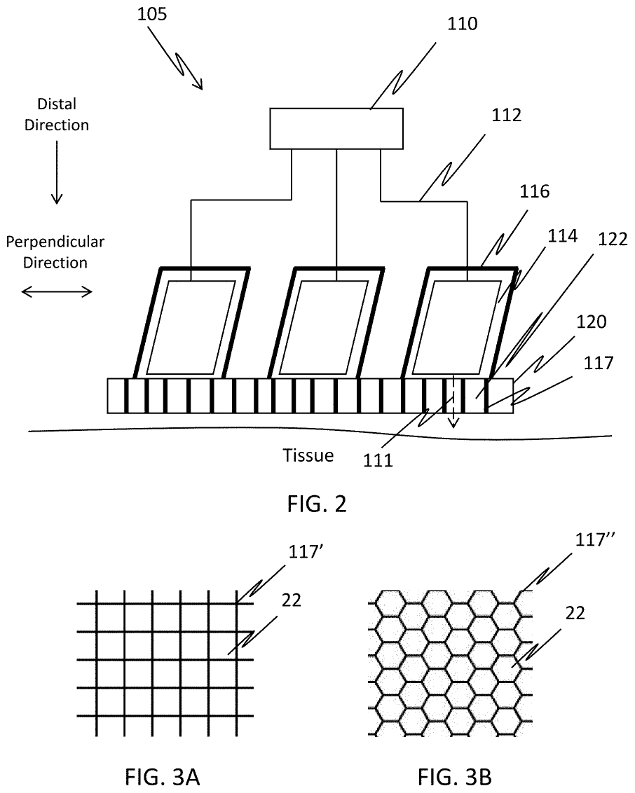

With reference now to FIG. 2, in one embodiment, an iontophoretic device 105 includes a driver 110 and drive lines 112 connected to separate electrodes 114 conductively isolated my a resistive barrier material 116. An inert resistive barrier material 117 is incorporated into the medication reservoir 120 forming chambers 122 to retain the medication in its aqueous or gel form. A mesh of resistive barrier material 117 forms one or more reservoir chambers 122 corresponding to each electrode 114 in the form of open cells that are interposed between the electrodes 114 and contact tissue. The conductive path between the electrode 114 and tissue via the corresponding chamber(s) 122 provides a current polarizing effect by providing a least resistive current flow path 111 running from the active electrode directly to the tissue contact point most proximal to the active electrode. The cells 122 retain fluid or gel medication by virtue of surface tension and hydrostatic effects. The cell walls 117 function to offer greater current resistance to lateral flow across the part of the medication reservoir 120 encompassing the cell wall structure. The cell configuration can have various geometries, including for example the rectangular or hexagonal configurations shown in FIGS. 3A and 3B respectively formed by a resistive barrier material 117′, 117″. In one embodiment, a porous mesh of nylon or polyamide (e.g. the types used for example in liquid filtration and particle entrapment applications) is utilized to form the cells.

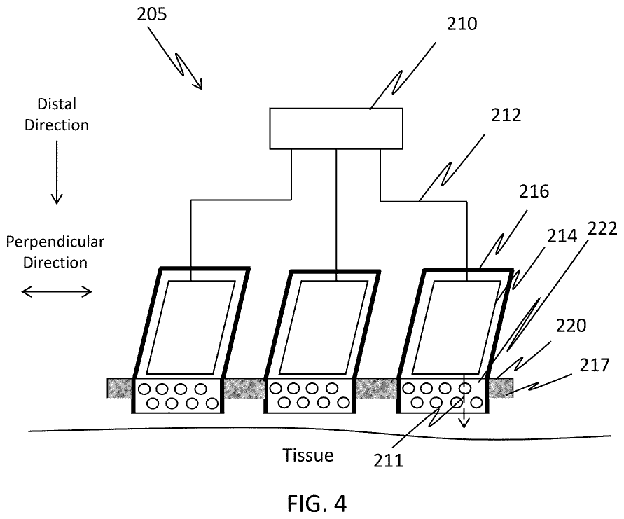

With reference now to FIG. 4, in one embodiment, an iontophoretic device 205 includes a driver 210 and drive lines 212 connected to separate electrodes 214 conductively isolated my a resistive barrier material 216. The medication reservoir 220 includes a cell foam or sponge material forming chambers 222 to retain the medication in its aqueous or gel form. Resistive barrier barriers 217 between the chamber sections 222 can be made from collapsed cells through a heat compression stamp process. The conductive path between the electrode 214 and tissue via the corresponding chamber 222 provides a current polarizing effect by providing a least resistive current flow path 211 running from the active electrode directly to the tissue contact point most proximal to the active electrode.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

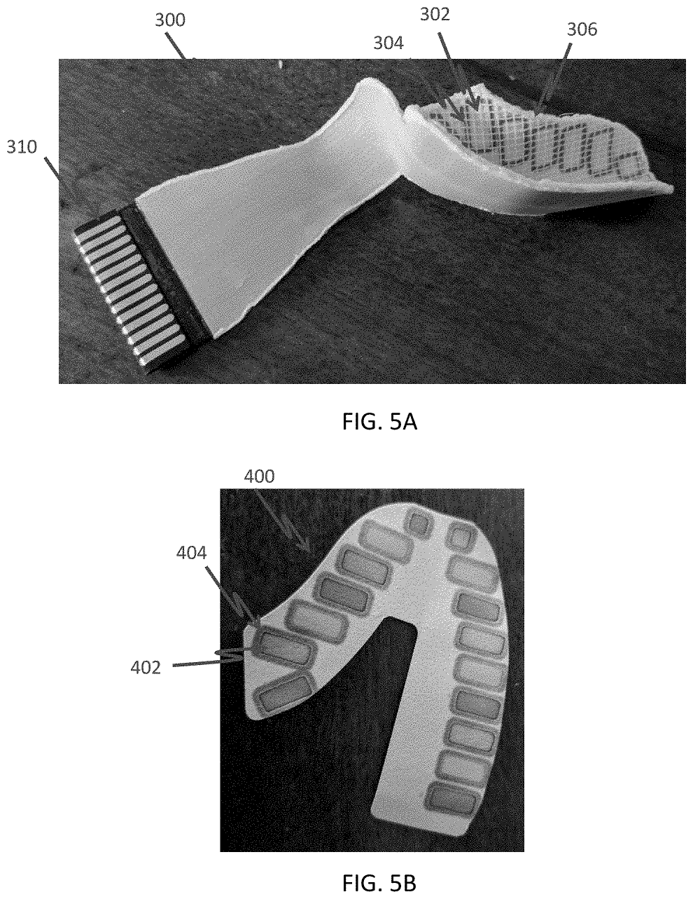

As shown in FIG. 5A, a dental application prototype 300 is shown according to one embodiment. The applicator includes channel driver contacts 310 that are electrically coupled to the electrodes 302. The electrodes 302 are conductively isolated by a resistive barrier layer 304 that runs along a perimeter of the electrode 302. A resistive barrier mesh layer 306 is placed adjacent to the electrodes 302 to direct the current to the closest treatment region. In the prototype 400 shown in FIG. 5B, the electrodes 402 each have a resistive barrier layer 404 shown in an alternate configuration. Applicator devices can be pre-formed or customized to fit the anatomy of the part of the body being treated. It should be appreciated that different electrodes can have different sizes, shapes and orientations. Different electrodes can also drive different current levels, based on the desired treatment or signals from a feedback loop.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. An iontophoretic device comprising:
a multichannel driver connected to a plurality of electrodes;
a medication reservoir positioned distal to the plurality of electrodes and comprising a plurality of reservoir chambers; and
a resistive barrier material at least partially surrounding one or more of the plurality of electrodes and one or more of the plurality of reservoir chambers, the resistive barrier material configured to provide a least resistive current flow path running from an active electrode to a tissue contact point most proximal to the active electrode.

2. The device of claim 1, wherein the resistive barrier material is a first material at least partially surrounding the one or more of the plurality of electrodes and second material at least partially surrounding the one or more of the plurality of reservoir chambers.

3. The device of claim 1, wherein the plurality of electrodes are separated by the resistive barrier material.

4. The device of claim 1, wherein the resistive barrier material is configured to allow current flow in a distal direction.

5. The device of claim 4, wherein the resistive barrier material is configured to block current flow in a direction perpendicular to the distal direction.

6. The device of claim 1, wherein each of the plurality of reservoir chambers are positioned directly below a different electrode.

7. The device of claim 1, wherein each of the plurality of electrodes are configured to direct a current flow path through a single reservoir chamber before reaching a target treatment area.

8. The device of claim 1, wherein the multichannel driver is configured to drive 0.6 milliamps or less of current through each channel.

9. The device of claim 1, wherein the resistive barrier material is disposed within the medication reservoir such that during application, a flow path from an active electrode to a tissue contact point most proximal to an adjacent electrode is resistive.

10. The device of claim 1, wherein the resistive barrier material is disposed within the medication reservoir such that during application, an inter-electrode flow path and an inter-channel flow path are both resistive.

11. The device of claim 1, wherein the medication reservoir is constructed from a resistive barrier mesh comprising open cells configured to hold medication in form of a liquid, gel, or ointment.

12. The device of claim 1, wherein the medication reservoir comprises a plurality of layers of open cell mesh.

13. The device of claim 1, wherein the medication reservoir comprises a polymer open cell structure.

14. The device of claim 1, wherein the medication reservoir comprises fibers with wicking properties.

15. The device of claim 1, wherein inter-channel spaces are rendered resistive by at least one of thermal cell collapse, resistive geometric infiltration or resistive structural isolation.
